(19) United States
(12) Patent Application Publication
(10) Pub. No.: US 2022/0297577 A1
(43) Pub. Date: Sep. 22, 2022

(54) Method, Computer Program Product and System for Mitigating Motion Sickness of a Vehicle Occupant, and a Motor Vehicle Equipped With Same

(71) Applicant: Bayerische Motoren Werke Aktiengesellschaft, Muenchen (DE)

(72) Inventors: Maximilian AMERELLER, Muenchen (DE); Georg BURKHARD, Muenchen (DE); Martin ENDERS, Deisenhofen (DE); Sonja RUEMELIN, Muenchen (DE); Elisabeth SCHMIDT, Muenchen (DE); Sascha WILHELM, Wolnzach (DE)

(21) Appl. No.: 17/596,583

(22) PCT Filed: Jun. 5, 2020
(86) PCT No.: PCT/EP2020/065689

§ 371 Date: Dec. 14, 2021

(30) Foreign Application Priority Data
Jun. 18, 2019 (DE) 10 2019 116 445.1

(51) Int. Cl. B60N 2/02; B60N 2/00; B60N 2/04; B60N 2/22; B60N 2/75; B60N 2/806; B60N 3/06; B60R 21/015; A61M 21/02

(52) U.S. Cl. CPC B60N 2/0276; B60N 2/002; B60N 2/04; B60N 2/22; B60N 2/763; B60N 2/806; B60N 3/063; B60R 21/015; A61M 21/02; B60N 2002/026; B60N 2002/0268

(57) ABSTRACT

A motion sickness mitigation method for the purposes of mitigating motion sickness of a vehicle occupant travelling in a motor vehicle includes moving at least a portion of a backrest, a portion of a seat squab, a portion of a head restraint, a portion of an armrest and/or a portion of a footrest as a function of a detected and/or an anticipated driving situation of the motor vehicle, in each case proceeding from a starting position in at least one spatial direction. The movement is situational on the basis of an actual or anticipated trajectory of the motor vehicle.

### BACKGROUND AND SUMMARY OF THE INVENTION

The present invention relates to a motion sickness mitigation method for mitigating motion sickness of a vehicle occupant, to a computer program product for carrying out the method, to a motion sickness mitigation system for mitigating motion sickness of a vehicle occupant, and to a motor vehicle equipped with such a system.

To prevent a vehicle driver and/or their passengers, that is to say vehicle occupants of a motor vehicle, from suffering from, or being at risk of suffering from, motion sickness-induced malaise during a journey in the motor vehicle, there is the approach, known from CN 204472614 U, DE 10 2008 055 553 A1 and DE 10 2008 055 554 A1, of varying the seat position of the vehicle occupant situated in the moving vehicle. Unfortunately, however, in pursuing this approach, it is disadvantageously not possible to rule out that desired mitigation occurs even in the case of vehicle occupants particularly sensitive to motion sickness.

It is an object of the present invention to specify technical ways with which the above-stated disadvantage of the prior art can be overcome.

Said object is achieved by way of a motion sickness mitigation method for mitigating motion sickness of a vehicle occupant situated in a motor vehicle according to the claimed invention. Note that the motion sickness mitigation method is also configured for preventing the occurrence of motion sickness entirely where possible.

Consequently, the stated object is achieved by way of a motion sickness mitigation method for mitigating motion sickness of a vehicle occupant situated in a motor vehicle, the upper body and lower body of which vehicle occupant are at least partially and at least intermittently supported by at least one backrest and/or one seat surface of a vehicle seat, the head of which vehicle occupant is at least partially and at least intermittently supported by a head restraint, of which vehicle occupant at least one arm is at least partially and at least intermittently supported by an armrest, and/or of which vehicle occupant at least one foot is at least partially and at least intermittently supported by a footrest, wherein at least a portion of the backrest, a portion of the seat surface, a portion of the head restraint, a portion of the armrest and/or a portion of the footrest is moved in each case from an initial position in at least one spatial direction in a manner dependent on a detected and/or anticipated driving situation of the motor vehicle. This movement is performed in a manner dependent on an anticipated or existing driving situation in accordance with one of the following steps, a combination of several of the following steps, or a combination of all of the following steps:

If the driving situation corresponds to straight-ahead travel with an acceleration, the head restraint remains in the initial position, the backrest is moved into a first backrest position that differs from the initial position, the armrest is moved into a first armrest position that differs from the initial position, the seat surface remains in the initial position, and the footrest remains in its initial position.

If the driving situation corresponds to straight-ahead travel with an anticipated acceleration, the head restraint remains in the initial position, the backrest remains in its initial position, the armrest is moved into a second armrest position, the seat surface remains in the initial position, and the footrest remains in its initial position.

If the driving situation corresponds to straight-ahead travel with a deceleration, the head restraint remains in the initial position, the backrest remains in its initial position, the armrest is moved into a third armrest position, the seat surface remains in the initial position, and the footrest remains in its initial position.

If the driving situation corresponds to straight-ahead travel with an anticipated deceleration, the head restraint remains in the initial position, the backrest remains in its initial position, the armrest is moved into a fourth armrest position, the seat surface remains in the initial position, and the footrest remains in its initial position.

If the driving situation corresponds to straight-ahead travel with a lifting movement, the head restraint remains in the initial position, the backrest remains in its initial position, the armrest is moved into a fifth armrest position, the seat surface is moved into a first seat surface position, and the footrest remains in its initial position.

If the driving situation corresponds to straight-ahead travel with a pitching movement, the head restraint is moved into a first head restraint position, the backrest is moved into a second head restraint position, the armrest is moved into a sixth armrest position, the seat surface remains in the initial position, and the footrest is moved into a first footrest position.

If the driving situation corresponds to straight-ahead travel with a rolling movement, the head restraint remains in the initial position, the backrest remains in its initial position, the armrest is moved into a seventh armrest position, the seat surface is moved into a second seat surface position, and the footrest is moved into a second footrest position.

If the driving situation corresponds to straight-ahead travel with a combination of acceleration, deceleration, lifting movement, pitching movement and rolling movement, the head restraint is moved into a second head restraint position, the backrest is moved into a third backrest position, the armrest is moved into an eighth armrest position, the seat surface is moved into a third seat surface position, and the footrest is moved into a third footrest position.

It is self-evident that the above-stated steps may be carried out individually or in any desired combination with one another in the method. Note also that the expression “mitigation of motion sickness” can also encompass the prevention thereof. Consequently, the method according to an embodiment of the invention encompasses a method for preventing and/or mitigating motion sickness in a motor vehicle. Note finally that the expression “movement” of a head restraint, of a backrest, of an armrest, of a seat surface or of a footrest is to be understood to mean not only an adjustment of the respectively stated element as a whole but also an adjustment of only a part or subregion, in particular of a side bolster, of the respectively stated element.

For the sake of clarity, the relationships disclosed above will be summarized once again in the following table 1:

TABLE 1

| Driving situation during | | Movement of the | Movement of | Movement of the | Movement of the | Movement of |
|---|---|---|---|---|---|---|
| straight-ahead travel | | head restraint | the backrest | armrest | seat surface | the footrest |
| 1. | Acceleration | None | First backrest | First armrest position | None | None |
| | | | position | | | |
| 2. | Anticipated | None | None | Second armrest | None | None |
| | acceleration | | | position | | |
| 3. | Deceleration | None | None | Third armrest position | None | None |
| 4. | Anticipated | None | None | Fourth armrest | None | None |
| | deceleration | | | position | | |
| 5. | Lifting movement | None | None | Fifth armrest position | First seat surface | None |
| | | | | | position | |
| 6. | Pitching movement | First head | Second backrest | Sixth armrest position | None | First footrest |
| | | restraint position | position | | | position |
| 7. | Rolling movement | None | None | Seventh armrest | Second seat | Second footrest |
| | | | | position | surface position | position |
| 8. | Mixture of 1. to 7. | Second head | Third backrest | Eighth armrest | Third seat | Third footrest |
| | | restraint position | position | position | surface position | position |

An embodiment of the present invention is advantageously distinguished by a holistic approach for mitigating or preventing motion sickness, in the case of which a large number of adjustment possibilities or movement possibilities of a head restraint, backrest, armrest, seat surface and footrest which are situated in a motor vehicle and which are provided for supporting a vehicle occupant are linked with one another as disclosed above in a manner dependent on a driving state of the motor vehicle.

The movements of head restraint, backrest, armrest, seat surface and/or footrest as disclosed above take place during straight-ahead travel of a correspondingly configured motor vehicle.

In accordance with the following advantageous refinements of the present invention, it is possible to also achieve mitigation of motion sickness during a cornering maneuver.

For this purpose, in the case of a steady-state cornering maneuver, the head restraint is moved into a third head restraint position, the backrest remains in its initial position, the armrest is moved into a ninth armrest position, the seat surface is moved into a fourth seat surface position, and the footrest is moved into a fourth footrest position.

Furthermore, in the case of a cornering maneuver with an entry to a destination, it may alternatively or additionally be provided that the head restraint is moved into a fourth head restraint position, the backrest remains in its initial position, the armrest is moved into a tenth armrest position, the seat surface is moved into a fifth seat surface position, and the footrest is moved into a fifth footrest position.

It may advantageously be provided that, in the case of a cornering maneuver with an exit to a destination, the head restraint is moved into a fifth head restraint position, the backrest remains in its initial position, the armrest is moved into an eleventh armrest position, the seat surface is moved into a sixth seat surface position, and the footrest is moved into a sixth footrest position.

Finally, in the case of an anticipated cornering maneuver, it may advantageously be provided that the head restraint remains in its initial position, the backrest remains in its initial position, the armrest is moved into a twelfth armrest position, the seat surface remains in its initial position, and the footrest remains in its initial position.

It is self-evident that a large number of different driving situations will arise during the course of a journey of a motor vehicle. The method according to an embodiment of the invention takes this fact into consideration and is advantageously configured such that the head restraint, backrest, armrest, seat surface and/or footrest assume their initial position, or are adjusted or moved, in a manner dependent on the scenarios presented.

It is the case here, too, that the expression “movement” of a head restraint, of a backrest, of an armrest, of a seat surface or of a footrest is to be understood to mean not only an adjustment of the respectively stated element as a whole but also an adjustment of only a part or subregion, in particular of a side bolster, of the respectively stated element.

For the sake of clarity, the relationships disclosed above and relating to a cornering maneuver will be summarized once again in the following table 2:

TABLE 2

| Driving situation during | | Movement of the | Movement of | Movement of | Movement of | Movement of |
|---|---|---|---|---|---|---|
| cornering maneuver | | head restraint | the backrest | the armrest | the seat surface | the footrest |
| 1. | Steady-state travel | Third head | None | Ninth armrest | Fourth seat | Fourth footrest |
| | (right-hand or left- | restraint position | | position | surface position | position |
| | hand corner) | | | | | |
| 2. | Entry to destination | Fourth head | None | Tenth armrest | Fifth seat surface | Fifth footrest |
| | | restraint position | | position | position | position |
| 3. | Exit to destination | Fifth head | None | Eleventh armrest | Sixth seat surface | Sixth footrest |
| | | restraint position | | position | position | position |
| 4. | Anticipated cornering | None | None | Twelfth armrest | None | None |
| | maneuver | | | position | | |

According to one preferred embodiment of the present invention, it is provided that the movement speed, the acceleration and/or the movement travel of the head restraint, proceeding from the initial position, for assuming the fourth head restraint position and/or fifth head restraint position is lower/smaller than that for assuming the first, second and/or third head restraint position. In this way, the risk of an occurrence of motion sickness, or the severity thereof, is advantageously reduced. For the sake of clarity, this relationship will be summarized briefly in the following table 3:

TABLE 3

| | | Movement speed, acceleration and/or |
|---|---|---|
| Driving situation during straight-ahead travel | | movement travel of the head restraint |
| 1. | Acceleration | None |
| 2. | Anticipated acceleration | None |
| 3. | Deceleration | None |
| 4. | Anticipated deceleration | None |
| 5. | Lifting movement | None |
| 6. | Pitching movement | Selected appropriately for this driving |
| | | situation. |
| 7. | Rolling movement | None |
| 8. | Mixture of 1. to 7. | Selected appropriately for this driving |
| | | situation. |
| Driving situation during cornering maneuver | | |
| 9. | Steady-state travel (right-hand or left- | Selected appropriately for this driving |
| | hand corner) | situation. |
| 10. | Entry to destination | Lower/smaller than in driving situation 6, 7 |
| | | and/or 8. |
| 11. | Exit to destination | Lower/smaller than in driving situation 6, 7 |
| | | and/or 8. |
| 12. | Anticipated cornering maneuver | None |

According to a further embodiment of the present invention, it is provided that the movement speed, the acceleration and/or the movement travel of the backrest, proceeding from the initial position, for assuming the second backrest position is lower/smaller than that for assuming the first and/or third backrest position. In this way, the risk of an occurrence of motion sickness, or the severity thereof, is advantageously reduced. For the sake of clarity, this relationship will be summarized once again in the following table 4:

TABLE 2

| | | Movement speed, acceleration and/or |
|---|---|---|
| Driving situation during straight-ahead travel | | movement travel of the backrest |
| 1. | Acceleration | Selected appropriately for this driving |
| | | situation. |
| 2. | Anticipated acceleration | None |
| 3. | Deceleration | None |
| 4. | Anticipated deceleration | None |
| 5. | Lifting movement | None |
| 6. | Pitching movement | Lower/smaller than in driving situation 1. |
| 7. | Rolling movement | None |
| 8. | Mixture of 1. to 7. | Selected appropriately for this driving |
| | | situation. |
| Driving situation during cornering maneuver | | |
| 9. | Steady-state travel (right-hand or left- | None |
| | hand corner) | |
| 10. | Entry to destination | None |
| 11. | Exit to destination | None |
| 12. | Anticipated cornering maneuver | None |

Alternatively or in addition, it may be provided that the movement speed, the acceleration and/or the movement travel of the armrest, proceeding from the initial position, for assuming the third, seventh, eighth and/or ninth armrest position is higher/greater than that for assuming the sixth, tenth and/or eleventh armrest position, the movement speed, acceleration and/or the movement travel of which in turn, proceeding from the initial position, are/is higher/greater than that for assuming the first and/or fifth armrest position. In this way, the risk of an occurrence of motion sickness, or the severity thereof, is advantageously further reduced. For the sake of clarity, this relationship will be summarized once again in the following table 5:

TABLE5

| | | Movement speed, acceleration and/or |
|---|---|---|
| Driving situation during straight-ahead travel | | movement travel of the armrest |
| 1. | Acceleration | Selected appropriately for this driving |
| | | situation. |
| 2. | Anticipated acceleration | Higher/greater than in driving situation 1. |
| 3. | Deceleration | Higher/greater than in driving situation 2. |
| 4. | Anticipated deceleration | Corresponds to driving situation 2. |
| 5. | Lifting movement | Corresponds to driving situation 1. |
| 6. | Pitching movement | Selected appropriately for this driving situation. |
| 7. | Rolling movement | Corresponds to driving situation 3. |
| 8. | Mixture of 1. to 7. | Corresponds to driving situation 3. |
| Driving situation during cornering maneuver | | |
| 9. | Steady-state travel (right-hand or left- | Corresponds to driving situation 3. |
| | hand corner) | |
| 10. | Entry to destination | Corresponds to driving situation 6. |
| 11. | Exit to destination | Corresponds to driving situation 6. |
| 12. | Anticipated cornering maneuver | Selected appropriately for this driving |
| | | situation. |

Alternatively or in addition, it may be provided that the movement speed, the acceleration and/or the movement travel of the seat surface, proceeding from the initial position, for assuming the third seat surface position is higher/greater than that for assuming the fourth seat surface position, the movement speed, acceleration and/or the movement travel of which in turn, proceeding from the initial position, are/is higher/greater than that for assuming the first, fifth and/or sixth armrest position. In this way, the risk of an occurrence of motion sickness, or the severity thereof, is advantageously further reduced. For the sake of clarity, this relationship will be summarized once again in the following table 6:

TABLE 6

| | | Movement speed, acceleration and/or |
|---|---|---|
| Driving situation during straight-ahead travel | | movement travel of the seat surface |
| 1. | Acceleration | None |
| 2. | Anticipated acceleration | None |
| 3. | Deceleration | None |
| 4. | Anticipated deceleration | None |
| 5. | Lifting movement | Selected appropriately for this driving |
| | | situation. |
| 6. | Pitching movement | None |
| 7. | Rolling movement | Corresponds to driving situation 5. |
| 8. | Mixture of 1. to 7. | Higher/greater than in driving situation 5. |
| Driving situation during cornering maneuver | | |
| 9. | Steady-state travel (right-hand or left- | Higher/greater than in driving situation 5 |
| | hand corner) | and lower/smaller than in driving situation |
| | | 8. |
| 10. | Entry to destination | Corresponds to driving situation 5. |
| 11. | Exit to destination | Corresponds to driving situation 5. |
| 12. | Anticipated cornering maneuver | None |

Alternatively or in addition, it may be provided that the movement speed, the acceleration and/or the movement travel of the footrest, proceeding from the initial position, for assuming the first, second, third and/or fourth footrest position is higher/greater than that for assuming the fifth and/or sixth footrest position. For the sake of clarity, this relationship will be summarized once again in the following table 7:

TABLE 7

| | | Movement speed, acceleration and/or |
|---|---|---|
| Driving situation during straight-ahead travel | | movement travel of the footrest |
| 1. | Acceleration | None |
| 2. | Anticipated acceleration | None |
| 3. | Deceleration | None |
| 4. | Anticipated deceleration | None |
| 5. | Lifting movement | None |
| 6. | Pitching movement | Selected appropriately for this driving |
| | | situation. |
| 7. | Rolling movement | Corresponds to driving situation 6. |
| 8. | Mixture of 1. to 7. | Corresponds to driving situation 6. |
| Driving situation during cornering maneuver | | |
| 9. | Steady-state travel (right-hand or left- | Corresponds to driving situation 6. |
| | hand corner) | |
| 10. | Entry to destination | Corresponds to driving situation 6. |
| 11. | Exit to destination | Corresponds to driving situation 6. |
| 12. | Anticipated cornering maneuver | None |

A movement of head restraint, backrest, armrest, seat surface or footrest for assuming its respective initial position, the first head restraint position, the second head restraint position, the third head restraint position, the fourth head restraint position, the fifth head restraint position, the first backrest position, the second backrest position, the third backrest position, the first armrest position, the second armrest position, the third armrest position, the fourth armrest position, the fifth armrest position, the sixth armrest position, the seventh armrest position, the eighth armrest position, the ninth armrest position, the tenth armrest position, the eleventh armrest position, the first seat surface position, the second seat surface position, the third seat surface position, the fourth seat surface position, the fifth seat surface position, the sixth seat surface position, the first footrest position, the second footrest position, the third footrest position, the fourth footrest position, the fifth footrest position or the sixth footrest position may advantageously take place in a temporally and/or spatially linear or non-linear, in particular translational and/or rotational, manner. In this way, the method according to an embodiment of the invention can advantageously be adapted to any driving situations.

In a further embodiment according to the invention, it is provided that the movement speed and/or the acceleration and/or the movement travel and/or the movement duration of the head restraint, of the backrest, of the armrest, of the seat surface and/or of the footrest is at least partially opposed to the detected and/or anticipated movement speed and/or the acceleration and/or the movement travel and/or the movement duration of the motor vehicle. Consequently, the movement direction and/or the movement speed and/or the movement acceleration and/or the movement duration of the respectively moved part of the head restraint, of the backrest, of the armrest, of the seat surface and/or of the footrest is at least partially opposed to the actual movement direction and/or the movement speed and/or the movement acceleration and/or the movement duration of the motor vehicle. In this way, it can be achieved with even greater likelihood that motion sickness is mitigated or prevented.

The above-stated object is likewise achieved by way of a computer program product which is stored in a computer-readable medium and which has computer-readable program code by which a computer as a data processing arrangement and/or a cloud-based data processing arrangement are caused to carry out a method of the above-disclosed type if the computer program product is executed on the computer and/or the cloud-based data processing arrangement. The above-stated advantages apply appropriately.

The above-stated object is likewise achieved by way of a motion sickness mitigation system for mitigating motion sickness of a vehicle occupant situated in a motor vehicle, wherein at least one backrest and at least one seat surface of at least one vehicle seat, at least one head restraint, at least one armrest and/or at least one footrest are situated in the motor vehicle, and wherein the backrest, the seat surface, the head restraint, the armrest and/or the footrest are operatively connected to at least one adjuster, in particular to an electrical, electromechanical, pneumatic or hydraulic actuator, which in turn is operatively connected to at least one data processing arrangement on which the computer program product of the above-disclosed type is executed. The above-stated advantages apply appropriately. It is self-evident that, according to an embodiment of the invention, it is also possible for a controller to be provided which is operatively connected both to the data processing arrangement and to the adjuster. Furthermore, a detector for detecting seat occupancy of one, several or all vehicle seats may be provided, such that the method according to an embodiment of the invention is carried out only if a vehicle occupant is situated on a vehicle seat. The detector for detecting seat occupancy may be situated in the backrest, the seat surface, the head restraint, the armrest and/or the footrest.

It is pointed out that the adjustment of the head restraint, of the backrest, of the armrest, of the seat surface and of the footrest of the motion sickness mitigation system according to an embodiment of the invention is performed with the aid of at least one adjustor such that at least one part of the head restraint, of the backrest, of the armrest, of the seat surface and of the footrest (in particular if these are formed in the manner of a side bolster or seat bolster) is adjusted in a freely selectable direction in space. Consequently, the adjustment of the respective elements stated above may take place in any spatial direction by way of a rotation, a translation or a combination of translation with rotation. A triggering of the adjustment, the ending thereof and the nature thereof, in particular direction, speed, acceleration and/or duration, is performed in a manner dependent, or is dependent, on a detected driving situation of the motor vehicle and/or on an anticipated driving situation.

Consequently, a motion sickness mitigation system for mitigating motion sickness of a vehicle occupant situated in a motor vehicle is advantageously created, which has a high number of adjustment possibilities in relation to the prior art and thus, in the truest sense of the word, has a positively noticeable effect on vehicle occupants afflicted with motion sickness.

According to one preferred embodiment, the device according to the invention has at least one detector for detecting the physical or psychological state of the vehicle occupant (in particular their heart rate, the nature of the movement of their eyes and/or their secretion of sweat), which at least one detector is operatively connected to a controller, which is operatively connected to the data processing arrangement and to the adjustor. In this way, it is advantageously possible for the severity of the motion sickness or the state of the vehicle occupant to be detected in situ in order—in a manner dependent on this—to adjust the head restraint, the backrest, the armrest, the seat surface and the footrest of the motion sickness mitigation system according to an embodiment of the invention. It is advantageously thus the case that the triggering of the adjustment, the ending thereof and the nature thereof, in particular direction, speed, acceleration and/or duration, is performed in a manner dependent, or is dependent, not only on a detected driving situation of the motor vehicle and/or on an anticipated driving situation but also on the physical and/or psychological state of the vehicle occupant.

The above-stated object is likewise achieved by way of a motor vehicle that has at least one motion sickness mitigation system of the above-disclosed type. The above-stated advantages apply appropriately.

A detailed, non-prejudicial, in particular non-restrictive, description of an exemplary embodiment of the present invention will be given below with reference to the appended figures, which are not true to scale. Identical elements are denoted by identical reference designations unless stated otherwise.

### BRIEF DESCRIPTION OF THE DRAWINGS

FIG. 1 is a plan view of a schematically illustrated motor vehicle according to an embodiment of the invention, in which a system according to an embodiment of the invention for preventing and/or mitigating motion sickness is arranged.

FIG. 2 is a schematic view of an exemplary trajectory of the motor vehicle illustrated in FIG. 1.

### DETAILED DESCRIPTION OF THE DRAWINGS

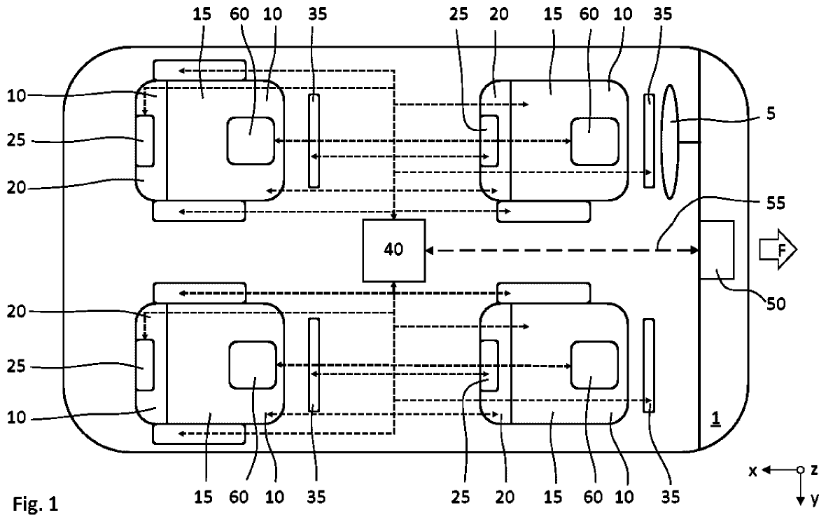

FIG. 1 schematically shows a motor vehicle 1 which—as can be seen from the symbolically illustrated steering wheel 5—is configured as a left-hand-drive vehicle and the main direction of travel of which is represented by the arrow denoted by F. A series of vehicle seats 10, specifically a driver's seat, a front passenger seat, a left-hand passenger seat arranged behind the driver's seat, and a right-hand passenger seat situated to the right of said left-hand passenger seat as viewed in the main direction of travel F, are situated in the motor vehicle 1.

Each of the vehicle seats 10 has, in a manner known per se, a seat surface 15, a backrest 20, a head restraint 25 and at least one armrest 30. As viewed in the direction of travel F, a footrest 35 known per se is additionally situated in front of each vehicle seat 10.

Each seat surface 15, backrest 20, head restraint 25, armrest 30 and footrest 35 comprises an adjustor, which for the sake of clarity of FIG. 1 is not shown. Using a single adjustor, several adjustors or all adjustors, it is possible to vary in particular the shape, the size and/or the position of the respective seat surface 15, backrest 20, head restraint 25, armrest 30 and footrest 35 in space, in particular to adjust same in translation and/or rotation relative to one, several or all axes of a vehicle-based Cartesian coordinate system x, y, z known per se and conforming to ISO 4130-1978, as will be discussed further below.

For the open-loop and/or closed-loop control of the adjustment, each adjustor is operatively connected to a controller 40 that outputs adjustment signals; according to this exemplary embodiment, all adjustors are operatively connected to a single controller 40, as symbolized by the double arrows shown in dashed lines and without reference designation. The controller 40 itself is operatively connected (cf. double arrow 55) to an on-board electronics system 50 known per se, which is situated in the motor vehicle and which, for the adjustment of the respective seat surface 15, backrest 20, head restraint 25, armrest 30 and/or footrest 35, outputs relevant data, in particular the speed of the motor vehicle 1, the steering angle, acceleration or deceleration and/or speed thereof, to the controller 40 after the movement speed and/or the acceleration and/or the movement travel and/or the movement duration has been calculated by way of a suitable computer program product.

According to this exemplary embodiment, on each vehicle seat 10, there is provided a detector 60 by way of which the physical and/or psychological state of vehicle occupants (not shown here) of the motor vehicle 1 can be determined, in particular on the basis of their heart rate or possibly secreted facial sweat. It is self-evidently not imperatively necessary for every vehicle seat 10 to be equipped with a dedicated detector 60. Rather, it is in particular also possible for detector 60 to be arranged such that mutually adjacently situated vehicle seats 10 can be monitored in pairwise fashion by in each case one single detector 60. Each detector 60 is operatively connected to the controller 40 such that not only the above-stated data representing the driving state of the motor vehicle 1 but also the data representing the physical and/or psychological state of the one or more vehicle occupants are taken into consideration in the control of each adjustor 30. It is thus in particular firstly possible for no motion-sickness-based adjustment of the respective seat surface 15, backrest 20, head restraint 25, armrest 30 and footrest 35 whatsoever to be performed in the case of vehicle occupants not suffering from motion sickness, and secondly for a seating-position-specific adjustment of the respective seat surface 15, backrest 20, head restraint 25, armrest 30 and footrest 35 to be performed, in the case of which a greater or lesser adjustment of the respective seat surface 15, backrest 20, head restraint 25, armrest 30 and footrest 35 is performed at the respective vehicle seat 10 in accordance with the severity of the motion sickness of the one or more vehicle occupants.

The one or more controllers 40 act(s) on the basis of data that are input in automated or automatic fashion by way of the on-board electronics system 50. It is furthermore possible for corresponding data that influence the adjustment of the respective seat surface 15, backrest 20, head restraint 25, armrest 30 and footrest 35 to be input manually by way of an input device that is not shown here. In this way, it is advantageously possible for a vehicle occupant to input motion-sickness-relevant data relating personally to themself or to other vehicle occupants in order for a desired adjustment of the respective seat surface 15, backrest 20, head restraint 25, armrest 30 and footrest 35 to be performed, or not performed, for themself or the other vehicle occupants. Alternatively or in addition, this input may be performed by way of an electronic data transmission device, in particular a smartphone, tablet computer or the like.

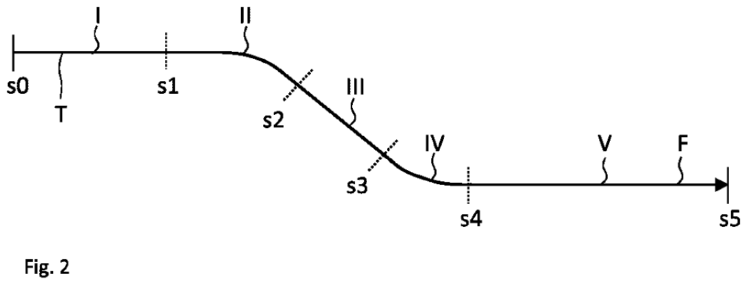

FIG. 2 illustrates an exemplary trajectory of a motor vehicle 1 according to an embodiment of the invention, which has been divided into five different route sections I, II, III, IV and V. Consequently, the motor vehicle 1 travels from a starting point s0 to an end point s1 on the route section I, from a route section II starting from the end point s1 to an end point s2, from a route section III starting from the end point s2 to an end point s3, from a route section IV starting from the end point s3 to an end point s4, and a route section V starting from the end point s4 to an end point s5.

In the straight route section I, the motor vehicle 1 accelerates from the starting point s0 to the end point s1. In this case, no movement of the head restraint 25, of the footrest 35 or of the seat surface 15 is performed, with only the backrest 20 being moved from an initial position into a first backrest position and the armrest 30 being moved from an initial position into a first armrest position. The first backrest position is adjusted partially downward (that is to say counter to the z direction) and forward (that is to say counter to the x direction) in relation to the initial position thereof. The first armrest position is adjusted likewise downward (that is to say counter to the z direction) and forward (that is to say counter to the x direction) in relation to the initial position thereof

In the second route section II, the motor vehicle I travels through a right-hand corner. The vehicle occupant is notified of this in advance at the end point s1, with the armrest 30 being adjusted, whilst the head restraint 25, the backrest 20, the seat surface 15 and the footrest 35 remain in their position. Specifically, the armrest situated on the left in the main direction of travel F is moved upward (that is to say in the z direction) and forward (that is to say in the x direction), whereas the armrest situated on the right in the main direction of travel F is moved downward and rearward (that is to say in the x direction).

The motor vehicle 1 subsequently travels through the corner of the route section II in steady-state fashion, that is to say with a constant speed. Here, the head restraint 25, the armrest 30, the seat surface 15 and the footrest 35 are moved, whilst the backrest remains in its position. The movement of the armrest 30 is more pronounced in this part of the driving maneuver of the motor vehicle 1 than upon the above-described advance notification of the cornering maneuver.

The motor vehicle 1 subsequently exits the corner, before traveling straight ahead again at the end point s2. During this exiting maneuver, the head restraint 25, the armrest 30, the seat surface 15 and the footrest 35 are moved, whilst the backrest 20 remains in its position. The magnitude of the movement of the head restraint 25 substantially corresponds here to the magnitude of its movement during the travel through the corner at constant speed. The magnitude of the movement of the armrest 30 in this route section is smaller than that during the travel through the corner at constant speed. The magnitude of the movement of the seat surface 15 in this route section is smaller than that during the travel through the corner at constant speed. The magnitude of the movement of the footrest 35 in this route section substantially corresponds to that during the travel through the corner at constant speed.

In the third route section III, the motor vehicle 1 travels straight ahead at constant speed on a flat roadway, with no automatic movement of head restraint 25, backrest 20, armrest 30, seat surface 15 or footrest 35 being performed.

Since a left-hand corner is to be traveled through in the route section IV following said route section III, the vehicle occupant is notified of a corner in advance at the end point s3. In this case, the armrests 30 are adjusted, whilst the head restraint 25, the backrest 20, the seat surface 15 and the footrest 35 remain in their position. Specifically, the armrest situated on the left in the main direction of travel F is moved downward and rearward, whereas the armrest situated on the right in the main direction of travel F is moved upward and forward.

After exiting the left-hand corner of the route section IV at the end point s4, the motor vehicle 1 travels straight ahead, wherein, in the case of the trajectory T considered here by way of example, said motor vehicle experiences firstly a lifting movement and subsequently a pitching movement before reaching an endpoint s5, which represents a complete standstill.

During the lifting movement, the armrest 30 and the seat surface 15 are moved, whilst the head restraint 25, the backrest 20 and the footrest 30 remain in position. During the pitching movement, the head restraint 25, the backrest 20, the armrest 30 and the footrest 35 are moved, whilst the seat surface 15 remains in position. The magnitude of the movement of the head restraint 25 during the lifting movement substantially corresponds to its magnitude during the steady-state cornering maneuver. The movement of the backrest 25 during the pitching movement is smaller than that during the acceleration during straight-ahead travel of the motor vehicle 1. The movement of the armrest 30 during the pitching movement is greater than that during the lifting movement or the acceleration of the motor vehicle 1 during straight-ahead travel. The magnitude of the movement of the seat surface 15 during the lifting movement is substantially equal to that during a rolling movement of the motor vehicle 1 or upon the exit of said motor vehicle from a corner with a destination. The magnitude of the movement of the footrest 35 corresponds substantially to that during a rolling movement of the motor vehicle 1 or upon the exit of said vehicle from a corner with a destination.

For the complete standstill of the motor vehicle 1 at the end point s5, the armrest 30 is moved, whilst the head restraint 25, the backrest 20, the seat surface 15 and the footrest remain in position. The magnitude of the movement of the armrest 30 is in this case greater than that during a lifting or pitching movement and substantially corresponds to that during a rolling movement of the motor vehicle 1.

### LIST OF REFERENCE DESIGNATIONS

1 Motor vehicle

5 Steering wheel

10 Vehicle seat

15 Seat surface

20 Backrest

25 Head restraint

30 Armrest

35 Footrest

40 Controller

50 On-board electronics system

55 Operative connection

60 Detector

I, II, III, IV, V Route sections

F Main direction of travel

T Trajectory

s1, s2, s3, s4, s5 Defined points on the trajectory

x, y, z Axes of a vehicle-based Cartesian coordinate system known per se, as per ISO 4130-1978

1.-15. (canceled)

16. A motion sickness mitigation method for mitigating motion sickness of a vehicle occupant situated in a motor vehicle, wherein the upper body and lower body of the vehicle occupant are at least partially and at least intermittently supported by at least one backrest and/or one seat surface of a vehicle seat, the head of the vehicle occupant is at least partially and at least intermittently supported by a head restraint, at least one arm of the vehicle occupant is at least partially and at least intermittently supported by an armrest, and/or at least one foot of the vehicle occupant is at least partially and at least intermittently supported by a footrest, wherein at least a portion of the backrest, a portion of the seat surface, a portion of the head restraint, a portion of the armrest and/or a portion of the footrest is moved from an initial position in at least one spatial direction in a manner dependent on a detected and/or anticipated driving situation of the motor vehicle, the motion sickness mitigation method comprising:
if the driving situation corresponds to straight-ahead travel with an acceleration, keeping the head restraint in the initial position, moving the backrest into a first backrest position that differs from the initial position, moving the armrest into a first armrest position that differs from the initial position, keeping the seat surface in the initial position, and keeping the footrest in the initial position, and/or
if the driving situation corresponds to straight-ahead travel with an anticipated acceleration, keeping the head restraint in the initial position, keeping the backrest in the initial position, moving the armrest into a second armrest position, keeping the seat surface in the initial position, and keeping the footrest in the initial position, and/or
if the driving situation corresponds to straight-ahead travel with a deceleration, keeping the head restraint in the initial position, keeping the backrest in the initial position, moving the armrest into a third armrest position, keeping the seat surface in the initial position, and keeping the footrest in the initial position, and/or
if the driving situation corresponds to straight-ahead travel with an anticipated deceleration, keeping the head restraint in the initial position, keeping the backrest in the initial position, moving the armrest into a fourth armrest position, keeping the seat surface in the initial position, and keeping the footrest in the initial position, and/or
if the driving situation corresponds to straight-ahead travel with a lifting movement, keeping the head restraint in the initial position, keeping the backrest in the initial position, moving the armrest into a fifth armrest position, moving the seat surface into a first seat surface position, and keeping the footrest in the initial position, and/or
if the driving situation corresponds to straight-head travel with a pitching movement, moving the head restraint into a first head restraint position, moving the backrest into a second backrest position, moving the armrest into a sixth armrest position, keeping the seat surface in the initial position, and moving the footrest into a first footrest position, and/or
if the driving situation corresponds to straight-ahead travel with a rolling movement, keeping the head restraint in the initial position, keeping the backrest in the initial position, moving the armrest into a seventh armrest position, moving the seat surface into a second seat surface position, and moving the footrest into a second footrest position, and/or
if the driving situation corresponds to straight-ahead travel with a combination of acceleration, deceleration, lifting movement, pitching movement and rolling movement, moving the head restraint into a second head restraint position, moving the backrest into a third backrest position, moving the armrest into an eighth armrest position, moving the seat surface into a third seat surface position, and moving the footrest into a third footrest position.

17. The motion sickness mitigation method according to claim 16, wherein if the driving situation corresponds to a steady-state cornering maneuver, the head restraint is moved into a third head restraint position, the backrest remains in the initial position, the armrest is moved into a ninth armrest position, the seat surface is moved into a fourth seat surface position, and the footrest is moved into a fourth footrest position.

18. The motion sickness mitigation method according to claim 16, wherein if the driving situation corresponds to a cornering maneuver with an entry to a destination, the head restraint is moved into a fourth head restraint position, the backrest remains in the initial position, the armrest is moved into a tenth armrest position, the seat surface is moved into a fifth seat surface position, and the footrest is moved into a fifth footrest position.

19. The motion sickness mitigation method according to claim 16, wherein if the driving situation corresponds to a cornering maneuver with an exit to a destination, the head restraint is moved into a fifth head restraint position, the backrest remains in the initial position, the armrest is moved into an eleventh armrest position, the seat surface is moved into a sixth seat surface position, and the footrest is moved into a sixth footrest position.

20. The motion sickness mitigation method according to claim 16, wherein if the driving situation corresponds to an anticipated cornering maneuver, the head restraint remains in the initial position, the backrest remains in the initial position, the armrest is moved into a twelfth armrest position, the seat surface remains in the initial position, and the footrest remains in the initial position.

21. The motion sickness mitigation method according to claim 16, wherein the movement speed, the acceleration and/or the movement travel of the head restraint, proceeding from the initial position, for assuming the fourth head restraint position and/or fifth head restraint position is smaller than the movement speed, the acceleration, and/or the movement travel of the head restraint for assuming the first, second and/or third head restraint position.

22. The motion sickness mitigation method according to claim 16, wherein the movement speed, the acceleration and/or the movement travel of the backrest, proceeding from the initial position, for assuming the second backrest position is smaller than the movement speed, the acceleration and/or the movement travel of the backrest for assuming the first and/or third backrest position.

23. The motion sickness mitigation method according to claim 16, wherein the movement speed, the acceleration and/or the movement travel of the armrest, proceeding from the initial position, for assuming the third, seventh, eighth and/or ninth armrest position is greater than the movement speed, the acceleration and/or the movement travel of the armrest for assuming the sixth, tenth and/or eleventh armrest position, and the movement speed, acceleration and/or the movement travel of the armrest, proceeding from the initial position, is greater than the movement speed, the acceleration and/or the movement travel of the armrest for assuming the first and/or fifth armrest position.

24. The motion sickness mitigation method according to claim 16, wherein the movement speed, the acceleration and/or the movement travel of the seat surface, proceeding from the initial position, for assuming the third seat surface position is greater than the movement speed, the acceleration and/or the movement travel of the seat surface for assuming the fourth seat surface position, and the movement speed, acceleration and/or the movement travel of the seat surface, proceeding from the initial position, is greater than the movement speed, the acceleration and/or the movement travel of the seat surface for assuming the first, fifth and/or sixth seat surface position.

25. The motion sickness mitigation method according to claim 16, wherein the movement speed, the acceleration and/or the movement travel of the footrest, proceeding from the initial position, for assuming the first, second, third and/or fourth footrest position is greater than the movement speed, the acceleration and/or the movement travel of the footrest for assuming the fifth and/or sixth footrest position.

26. The motion sickness mitigation method according to claim 16, wherein the head restraint, the backrest, the armrest, the seat surface and/or the footrest is moved in a temporally and/or spatially linear or non-linear manner.

27. The motion sickness mitigation method according to claim 16, wherein the movement speed and/or the acceleration and/or the movement travel and/or the movement duration of the head restraint, of the backrest, of the armrest, of the seat surface and/or of the footrest is at least partially opposed to the detected and/or anticipated movement speed and/or the acceleration and/or the movement travel and/or the movement duration of the motor vehicle.

28. A computer program product for mitigating motion sickness of a vehicle occupant situated in a motor vehicle, wherein the upper body and lower body of the vehicle occupant are at least partially and at least intermittently supported by at least one backrest and/or one seat surface of a vehicle seat, the head of the vehicle occupant is at least partially and at least intermittently supported by a head restraint, at least one arm of the vehicle occupant is at least partially and at least intermittently supported by an armrest, and/or at least one foot of the vehicle occupant is at least partially and at least intermittently supported by a footrest, wherein at least a portion of the backrest, a portion of the seat surface, a portion of the head restraint, a portion of the armrest and/or a portion of the footrest is moved from an initial position in at least one spatial direction in a manner dependent on a detected and/or anticipated driving situation of the motor vehicle, the computer program product comprising a non-transitory computer readable medium having stored thereon program code which, when executed on a processor, carries out a method of:
if the driving situation corresponds to straight-ahead travel with an acceleration, keeping the head restraint in the initial position, moving the backrest into a first backrest position that differs from the initial position, moving the armrest into a first armrest position that differs from the initial position, keeping the seat surface in the initial position, and keeping the footrest in the initial position, and/or
if the driving situation corresponds to straight-ahead travel with an anticipated acceleration, keeping the head restraint in the initial position, keeping the backrest in the initial position, moving the armrest into a second armrest position, keeping the seat surface in the initial position, and keeping the footrest in the initial position, and/or
if the driving situation corresponds to straight-ahead travel with a deceleration, keeping the head restraint in the initial position, keeping the backrest in the initial position, moving the armrest into a third armrest position, keeping the seat surface in the initial position, and keeping the footrest in the initial position, and/or
if the driving situation corresponds to straight-ahead travel with an anticipated deceleration, keeping the head restraint in the initial position, keeping the backrest in the initial position, moving the armrest into a fourth armrest position, keeping the seat surface in the initial position, and keeping the footrest in the initial position, and/or
if the driving situation corresponds to straight-ahead travel with a lifting movement, keeping the head restraint in the initial position, keeping the backrest in the initial position, moving the armrest into a fifth armrest position, moving the seat surface into a first seat surface position, and keeping the footrest in the initial position, and/or
if the driving situation corresponds to straight-head travel with a pitching movement, moving the head restraint into a first head restraint position, moving the backrest into a second backrest position, moving the armrest into a sixth armrest position, keeping the seat surface in the initial position, and moving the footrest into a first footrest position, and/or
if the driving situation corresponds to straight-ahead travel with a rolling movement, keeping the head restraint in the initial position, keeping the backrest in the initial position, moving the armrest into a seventh armrest position, moving the seat surface into a second seat surface position, and moving the footrest into a second footrest position, and/or
if the driving situation corresponds to straight-ahead travel with a combination of acceleration, deceleration, lifting movement, pitching movement and rolling movement, moving the head restraint into a second head restraint position, moving the backrest into a third backrest position, moving the armrest into an eighth armrest position, moving the seat surface into a third seat surface position, and moving the footrest into a third footrest position.

29. A motion sickness mitigation system for mitigating motion sickness of a vehicle occupant situated in a motor vehicle, the motion sickness mitigation system comprising:
at least one backrest and at least one seat surface of at least one vehicle seat, at least one head restraint, at least one armrest and/or at least one footrest are situated in the motor vehicle, and wherein the backrest, the seat surface, the head restraint, the armrest and/or the footrest that are operatively connected to at least one adjustor,
wherein the adjustor is operatively connected to at least one data processing arrangement on which the computer program product according to claim 28 is executed.

30. A motor vehicle comprising the motion sickness mitigation system according to claim 29.
